(19) United States
(12) Patent Application Publication
(10) Pub. No.: US 2005/0153340 A1
(43) Pub. Date: Jul. 14, 2005

(54) Human RNase H and compositions and uses thereof

(76) Inventors: Stanley T. Crooke, Carlsbad, CA (US); Walter F. Lima, San Diego, CA (US); Hongjiang Wu, Carlsbad, CA (US)

Correspondence Address: LICATA & TYRRELL P.C., 66 E. MAIN STREET, MARLTON, NJ, 08053, US

(21) Appl. No.: 11/005,069

(22) Filed: Dec. 6, 2004

Related U.S. Application Data
(63) Continuation of application No. 09/781,712, filed on Feb. 12, 2001, now abandoned.

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The present invention relates to methods for using mammalian RNase H, including human RNase H, and compositions thereof, particularly for reduction of a selected cellular RNA target via antisense technology. A novel human RNAse HII polypeptide and the polynucleotide sequence encoding it are also disclosed.

### INTRODUCTION

This application is a continuation of U.S. Ser. No. 09/781,712 filed Feb. 12, 2001, which is herein incorporated by reference in its entirety.

### FIELD OF THE INVENTION

The present invention relates to methods for using mammalian RNase H and compositions thereof, particularly for reduction of selected cellular RNA via antisense technology.

### BACKGROUND OF THE INVENTION

RNase H hydrolyzes RNA in RNA-DNA hybrids. This enzymatic activity was first identified in calf thymus but has subsequently been described in a variety of organisms (Stein, H. and Hausen, P., Science, 1969, 166, 393-395; Hausen, P. and Stein, H., Eur. J. Biochem., 1970, 14, 278-283). RNase H activity appears to be ubiquitous in eukaryotes and bacteria (Itaya, M. and Kondo K. Nucleic Acids Res., 1991, 19, 4443-4449; Itaya et al., Mol. Gen. Genet., 1991 227, 438-445; Kanaya, S., and Itaya, M., J. Biol. Chem., 1992, 267, 10184-10192; Busen, W., J. Biol. Chem., 1980, 255, 9434-9443; Rong, Y. W. and Carl, P. L., 1990, Biochemistry 29, 383-389; Eder et al., Biochimie, 1993 75, 123-126). Although RNases H constitute a family of proteins of varying molecular weight, nucleolytic activity and substrate requirements appear to be similar for the various isotypes. For example, all RNases H studied to date function as endonucleases, exhibiting limited sequence specificity and requiring divalent cations (e.g., Mg²⁺, Mn²⁺) to produce cleavage products with 5′ phosphate and 3′ hydroxyl termini (Crouch, R. J., and Dirksen, M. L., Nuclease, Linn, S, M., & Roberts, R. J., Eds., Cold Spring Harbor Laboratory Press, Plainview, N.Y. 1982, 211-241).

In addition to playing a natural role in DNA replication, RNase H has also been shown to be capable of cleaving the RNA component of certain oligonucleotide-RNA duplexes. While many mechanisms have been proposed for oligonucleotide mediated destabilization of target RNAs, the primary mechanism by which antisense oligonucleotides are believed to cause a reduction in target RNA levels is through this RNase H action. Monia et al., J. Biol. Chem., 1993, 266: 13, 14514-14522. In vitro assays have demonstrated that oligonucleotides that are not substrates for RNase H can inhibit protein translation (Blake et al., Biochemistry, 1985, 24, 6139-4145) and that oligonucleotides inhibit protein translation in rabbit reticulocyte extracts that exhibit low RNase H activity. However, more efficient inhibition was found in systems that supported RNase H activity (Walder, R. Y. and Walder, J. A., Proc. Nat'l Acad. Sci. USA, 1988, 85, 5011-5015; Gagnor et al., Nucleic Acid Res., 1987, 15, 10419-10436; Cazenave et al., Nucleic Acid Res., 1989, 17, 4255-4273; and Dash et al., Proc. Nat'l Acad. Sci. USA, 1987, 84, 7896-7900.

RNase HI from E. coli is the best-characterized member of the RNase H family. The 3-dimensional structure of E. coli RNase HI has been determined by x-ray crystallography, and the key amino acids involved in binding and catalysis have been identified by site-directed mutagenesis (Nakamura et al., Proc. Natl. Acad. Sci. USA, 1991, 88, 11535-11539; Katayanagi et al., Nature, 1990, 347, 306-309; Yang et al., Science, 1990, 249, 1398-1405; Kanaya et al., J. Biol. Chem., 1991, 266, 11621-11627). The enzyme has two distinct structural domains. The major domain consists of four a helices and one large a sheet composed of three antiparallel â strands. The Mg²⁺ binding site is located on the â sheet and consists of three amino acids, Asp-10, Glu-48, and Gly-11 (Katayanagi et al., Proteins: Struct., Funct., Genet., 1993, 17, 337-346). This structural motif of the Mg²⁺ binding site surrounded by â strands is similar to that in DNase I (Suck, D., and Oefner, C., Nature, 1986, 321, 620-625). The minor domain is believed to constitute the predominant binding region of the enzyme and is composed of an á helix terminating with a loop. The loop region is composed of a cluster of positively charged amino acids that are believed to bind electrostatistically to the minor groove of the DNA/RNA heteroduplex substrate. Although the conformation of the RNA/DNA substrate can vary from A-form to B-form depending on the sequence composition, in general RNA/DNA heteroduplexes adopt an A-like geometry (Pardi et al., Biochemistry, 1981, 20, 3986-3996; Hall, K. B., and Mclaughlin, L. W., Biochemistry, 1991, 30, 10606-10613; Lane et al., Eur. J. Biochem., 1993, 215, 297-306). The entire binding interaction appears to comprise a single helical turn of the substrate duplex. Recently the binding characteristics, substrate requirements, cleavage products and effects of various chemical modifications of the substrates on the kinetic characteristics of E. coli RNase HI have been studied in more detail (Crooke, S. T. et al., Biochem. J., 1995, 312, 599-608; Lima, W. F. and Crooke, S. T., Biochemistry, 1997, 36, 390-398; Lima, W. F. et al., J. Biol. Chem., 1997, 272, 18191-18199; Tidd, D. M. and Worenius, H. M., Br. J. Cancer, 1989, 60, 343; Tidd, D. M. et al., Anti-Cancer Drug Des., 1988, 3, 117.

In addition to RNase HI, a second E. coli RNase H, RNase HII, has been cloned and characterized (Itaya, M., Proc. Natl. Acad. Sci. USA, 1990, 87, 8587-8591). It is comprised of 213 amino acids while RNase HI is 155 amino acids long. E. coli RNase HII displays only 17% homology with E. coli RNase HI. An RNase H cloned from S. typhimurium differed from E. coli RNase HI in only 11 positions and was 155 amino acids in length (Itaya, M. and Kondo K., Nucleic Acids Res., 1991, 19, 4443-4449; Itaya et al., Mol. Gen. Genet., 1991, 227, 438-445). An enzyme cloned from S. cerevisae was 30% homologous to E. coli RNase HI (Itaya, M. and Kondo K., Nucleic Acids Res., 1991, 19, 4443-4449; Itaya et al., Mol. Gen. Genet., 1991, 227, 438-445).

Proteins that display RNase H activity have also been cloned and purified from a number of viruses, other bacteria and yeast (Wintersberger, U. Pharmac. Ther., 1990, 48, 259-280). In many cases, proteins with RNase H activity appear to be fusion proteins in which RNase H is fused to the amino or carboxy end of another enzyme, often a DNA or RNA polymerase. The RNase H domain has been consistently found to be highly homologous to E. coli RNase HI, but because the other domains vary substantially, the molecular weights and other characteristics of the fusion proteins vary widely.

In higher eukaryotes two classes of RNase H have so far been defined based on differences in molecular weight, effects of divalent cations, sensitivity to sulfhydryl agents and immunological cross-reactivity (Busen et al., Eur. J. Biochem., 1977, 74, 203-208). RNase HII enzymes (also called RNase H2, formerly called Type 1 RNase H) are reported to have molecular weights in the 68-90 kDa range, be activated by either Mn²⁺ or Mg²⁺ and be insensitive to sulfhydryl agents. In contrast, RNase HI enzymes (also called RNase H1, formerly called Type 2 RNase H) have been reported to have molecular weights ranging from 31-45 kDa, to require Mg²⁺, to be highly sensitive to sulfhydryl agents and to be inhibited by Mn²⁺ (Busen, W., and Hausen, P., Eur. J. Biochem., 1975, 52, 179-190; Kane, C. M., Biochemistry, 1988, 27, 3187-3196; Busen, W., J. Biol. Chem., 1982, 257, 7106-7108.).

An enzyme with Type 2 RNase H characteristics has been purified to near homogeneity from human placenta (Frank et al., Nucleic Acids Res., 1994, 22, 5247-5254). This protein has a molecular weight of approximately 33 kDa and is active in a pH range of 6.5-10, with a pH optimum of 8.5-9. The enzyme requires Mg²⁺ and is inhibited by Mn²⁺ and n-ethyl maleimide. The products of cleavage reactions have 3′ hydroxyl and 5′ phosphate termini.

Multiple mammalian RNases H have recently been cloned, sequenced and expressed. These include human RNase HI [Crooke et al., U.S. Pat. No. 6,001,653; Wu et al., Antisense Nucl. Acid Drug Des. 1998, 8: 53-61; Genbank accession no. AF039652; Cerritelli and Crouch, 1998, Genomics 53, 300-307; Frank et al., 1998, Biol. Chem. 379, 1407-1412], human RNase HII [(Frank et al., 1998, Proc. Natl. Acad. Sci. USA 95, 12872-12877)] and other mammalian RNases H (Cerritelli and Crouch, ibid.). The availability of purified RNase H has facilitated efforts to understand the structure of the enzyme, its distribution and the function(s) it may serve.

In the present invention, methods of using mammalian RNase H for reducing selected target RNA levels via an antisense mechanism are provided.

### SUMMARY OF THE INVENTION

The present invention is generally related to methods of using mammalian RNase H, especially human RNAse H, for reducing selected target RNA levels, particularly via an antisense mechanism. The present invention provides methods of promoting or eliciting antisense inhibition of expression of a target protein via use of mammalian RNase H, including human RNase HI and/or human RNase HII. Methods of screening for effective antisense oligonucleotides and of producing effective antisense oligonucleotides using mammalian RNase H are also provided.

Yet another object of the present invention is to provide methods for identifying agents which modulate activity and/or levels of mammalian RNase H. In accordance with this aspect, the polynucleotides and polypeptides of the present invention are useful for research, biological and clinical purposes. For example, the polynucleotides and polypeptides are useful in defining the interaction of mammalian RNase H and antisense oligonucleotides and identifying means for enhancing this interaction so that antisense oligonucleotides are more effective at inhibiting their target mRNA.

Yet another object of the present invention is to provide a method of prognosticating efficacy of antisense therapy of a selected disease which comprises measuring the level or activity of mammalian RNase H in a target cell of the antisense therapy. Similarly, oligonucleotides can be screened to identify those oligonucleotides which are effective antisense agents by measuring binding of the oligonucleotide to the mammalian RNase H.

The present invention also provides a polypeptide which has been identified as a novel human RNase HII by homology between the nucleic acid sequence encoding the amino acid sequence set forth as SEQ ID NO: 1 and known nucleic acid sequences of Caenorhabditis elegans, yeast and E. coli RNase HII as well as an EST deduced mouse RNase H homolog. A culture containing this nucleic acid sequence has been deposited as ATCC Deposit No. PTA-2897. Mutant forms and active fragments of this polypeptide are also included in the present invention.

The present invention also provides polynucleotides that encode this human RNase HII, vectors comprising nucleic acids encoding this human RNase HII, host cells containing such vectors, antibodies targeted to this human RNase HII, and nucleic acid probes capable of hybridizing to a nucleic acid encoding this human RNase HII polypeptide. Pharmaceutical compositions which include a human RNase HII polypeptide or a vector encoding a human RNase HII polypeptide are also provided. Antisense oligonucleotides and methods for inhibiting expression of human RNAse HII are also provided.

### BRIEF DESCRIPTION OF THE DRAWINGS

FIG. 1 provides a novel human RNase HII primary sequence (299 amino acids; SEQ ID NO: 1) and sequence comparisons with mouse (SEQ ID NO: 2), C. elegans (SEQ ID NO: 3), yeast (300 amino acids; SEQ ID NO: 4) and E. coli RNase HII (298 amino acids; SEQ ID NO: 5). Boldface type indicates amino acid residues identical to human. Uppercase letters above alignment indicate amino acid residues identically conserved among species; lower case letters above alignment indicate residues similarly conserved.

### DETAILED DESCRIPTION OF THE INVENTION

The present invention relates to methods for promoting antisense inhibition of a selected RNA target using mammalian RNase H, or for eliciting cleavage of a selected target via antisense. In the context of this invention, “promoting antisense inhibition” or “promoting inhibition of expression” of a selected RNA target, or of its protein product, means inhibiting expression of the target or enhancing the inhibition of expression of the target. In one preferred embodiment, the mammalian RNase H is a human RNase H. The RNase H may be an RNase HI or an RNase HII. In one embodiment of these methods, the mammalian RNase H is present in an enriched amount. In the context of this invention, “enriched” means an amount greater than would naturally be found. RNase H may be present in an enriched amount through, for example, addition of exogenous RNase H, through selection of cells which overexpress RNase H or through manipulation of cells to cause overexpression of RNase H. The exogenously added RNase H may be added in the form of, for example, a cellular or tissue extract (such as HeLa cell extract), a biochemically purified or partially purified preparation of RNase H, or a cloned and expressed RNase H polypeptide. In some embodiments of the methods of the invention, the mammalian RNase H has SEQ ID NO: 1, 6, 7, 8, 9, 10, or 11.

The present invention also relates to methods of screening oligonucleotides to identify active antisense oligonucleotides. The oligonucleotides may be present as a library or mixture of oligonucleotides. The methods involve contacting a mammalian RNase H, one or more oligonucleotides and an RNA target under conditions in which an oligonucleotide/RNA duplex is formed. The RNase H may be present in an enriched amount.

The present invention also relates to prognostic assays wherein levels of RNase H in a cell type can be used in predicting the efficacy of antisense oligonucleotide therapy in specific target cells. High levels of RNase H in a selected cell type are expected to correlate with higher efficacy as compared to lower amounts of RNase H in a selected cell type which may result in poor cleavage of the mRNA upon binding with the antisense oligonucleotide. For example, the HTB-11 neuroblastoma cell line displayed lower levels of RNase HII than some other malignant cell types. Accordingly, in this cell type it may be desired to use antisense compounds which do not depend on RNase H activity for their efficacy. Similarly, oligonucleotides can be screened to identify those which are effective antisense agents by contacting RNase H with an oligonucleotide and measuring binding of the oligonucleotide to the RNase H. Methods of determining binding of two molecules are well known in the art. For example, in one embodiment, the oligonucleotide can be radiolabeled and binding of the oligonucleotide to human RNase H can be determined by autoradiography. Alternatively, fusion proteins of human RNase H with glutathione-S-transferase or small peptide tags can be prepared and immobilized to a solid phase such as beads. Labeled or unlabeled oligonucleotides to be screened for binding to this enzyme can then be incubated with the solid phase. Oligonucleotides which bind to the enzyme immobilized to the solid phase can then be identified either by detection of bound label or by eluting specifically the bound oligonucleotide from the solid phase. Another method involves screening of oligonucleotide libraries for binding partners. Recombinant tagged or labeled human RNase H is used to select oligonucleotides from the library which interact with the enzyme. Sequencing of the oligonucleotides leads to identification of those oligonucleotides which will be more effective as antisense agents.

The modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the target. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of the target, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are hereinbelow identified as preferred embodiments of the invention. The target sites to which these preferred sequences are complementary are hereinbelow referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2=, 3= or 5= hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3= to 5= phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotri-esters, methyl and other alkyl phosphonates including 3=-alkylene phosphonates, 5′-alkylene phosphonates and chiral phosphonates, phosphinates, phosphoramidates including 3=-amino phosphoramidate and aminoalkylphosphoramidates, thionophosphoramidates, thionoalkylphosphonates, thionoalkylphosphotriesters, selenophosphates and borano-phosphates having normal 3=−5=linkages, 2=−5=linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃) —CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2=position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2=-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further prefered modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 3′ or 4′ carbon atom wherein n is 1 or 2. LNAs and preparation thereof are described in WO 98/39352 and WO 99/14226.

Other preferred modifications include 2′-methoxy(2′-O—CH₃), 2′-aminopropoxy(2′-OCH₂CH₂CH₂NH₂), 2′-allyl(2′-CH₂—CH═CH₂), 2′-O-allyl(2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2=−5=linked oligonucleotides and the 5′ position of 5′ terminal nucleotide. Oligonucleotides may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C/C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2EC (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Oligonucleotides, particularly chimeric oligonucleotides, designed to elicit target cleavage by RNase H, thus are generally more potent than oligonucleotides of the same base sequence which are not so optimized. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

RNase H, by definition, cleaves the RNA strand of an RNA-DNA duplex. In exploiting RNase H for antisense technology, the DNA portion of the duplex is generally an antisense oligonucleotide. Because native DNA oligonucleotides (2′ deoxy oligonucleotides with phosphodiester linkages) are relatively unstable in cells due to poor nuclease resistance, modified oligonucleotides are preferred for antisense. For example, oligodeoxynucleotides with phosphorothioate backbone linkages are often used. This is an example of a DNA-like oligonucleotide which is able to elicit RNase H cleavage of its complementary target RNA. Nucleic acid helices can adopt more than one type of structure, most commonly the A- and B-forms. It is believed that, in general, oligonucleotides which have B-form-like conformational geometry are “DNA-like” and will be able to elicit RNase H upon duplexation with an RNA target. Furthermore, oligonucleotides which contain a “DNA-like” region of B-form-like conformational geometry are also believed to be able to elicit RNase H upon duplexation with an RNA target.

The nucleotides for this B-form portion are selected to specifically include ribo-pentofuranosyl and arabino-pentofuranosyl nucleotides. 2′-Deoxy-erythro-pentofuranosyl nucleotides also have B-form geometry and elicit RNase H activity. While not specifically excluded, if 2′-deoxy-erythro-pentofuranosyl nucleotides are included in the B-form portion of an oligonucleotide of the invention, such 2′-deoxy-erythro-pentofuranosyl nucleotides preferably does not constitute the totality of the nucleotides of that B-form portion of the oligonucleotide, but should be used in conjunction with ribonucleotides or an arabino nucleotides. As used herein, B-form geometry is inclusive of both C2′-endo and O4′-endo pucker, and the ribo and arabino nucleotides selected for inclusion in the oligonucleotide B-form portion are selected to be those nucleotides having C2′-endo conformation or those nucleotides having O4′-endo conformation. This is consistent with Berger, et. al., Nucleic Acids Research, 1998, 26, 2473-2480, who pointed out that in considering the furanose conformations in which nucleosides and nucleotides reside, B-form consideration should also be given to a O4′-endo pucker contribution.

Preferred for use as the B-form nucleotides for eliciting RNase H are ribonucleotides having 2′-deoxy-2′-S-methyl, 2′-deoxy-2′-methyl, 2′-deoxy-2′-amino, 2′-deoxy-2′-mono or dialkyl substituted amino, 2′-deoxy-2′-fluoromethyl, 2′-deoxy-2′-difluoromethyl, 2′-deoxy-2′-trifluoromethyl, 2′-deoxy-2′-methylene, 2′-deoxy-2′-fluoromethylene, 2′-deoxy-2′-difluoromethylene, 2′-deoxy-2′-ethyl, 2′-deoxy-2′-ethylene and 2′-deoxy-2′-acetylene. These nucleotides can alternately be described as 2′-SCH₃ ribonucleotide, 2′-CH₃ ribonucleotide, 2′-NH₂ ribonucleotide 2′-NH(C₁-C₂ alkyl) ribonucleotide, 2′-N(C₁-C₂ alkyl)₂ ribonucleotide, 2′-CH₂F ribonucleotide, 2′-CHF₂ ribonucleotide, 2′-CF₃ ribonucleotide, 2′=CH₂ ribonucleotide, 2′=CHF ribonucleotide, 2′=CF₂ ribonucleotide, 2′-C₂H₅ ribonucleotide, 2′-CH═CH₂ ribonucleotide, 2′-C/CH ribonucleotide. A further useful ribonucleotide is one having a ring located on the ribose ring in a cage-like structure including 3′,O,4=-C-methyleneribonucleotides. Such cage-like structures will physically fix the ribose ring in the desired conformation.

Additionally, preferred for use as the B-form nucleotides for eliciting RNase H are arabino nucleotides having 2′-deoxy-2′-cyano, 2′-deoxy-2′-fluoro, 2′-deoxy-2′-chloro, 2′-deoxy-2′-bromo, 2′-deoxy-2′-azido, 2′-methoxy and the unmodified arabino nucleotide (that includes a 2′-OH projecting upwards towards the base of the nucleotide). These arabino nucleotides can alternately be described as 2′-CN arabino nucleotide, 2′-F arabino nucleotide, 2′-Cl arabino nucleotide, 2′-Br arabino nucleotide, 2′-N₃ arabino nucleotide, 2′-O—CH₃ arabino nucleotide and arabino nucleotide.

Such nucleotides are linked together via phosphorothioate, phosphorodithioate, boranophosphate or phosphodiester linkages. particularly preferred is the phosphorothioate linkage.

Illustrative of the B-form nucleotides for use in the invention is a 2′-S-methyl(2′-SMe) nucleotide that resides in C2′ endo conformation. It has been compared by molecular modeling to a 2′-O-methyl(2′-OMe)nucleotide that resides in a C3′ endo conformation.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

In accordance with one aspect of the present invention, there are provided isolated polynucleotides which encode human RNase HII polypeptides having the deduced amino acid sequence of SEQ ID NO: 1. A culture containing this nucleic acid sequence has been deposited as ATCC Deposit No. PTA-2897. “Polynucleotides” is meant to include any form of RNA or DNA such as mRNA or cDNA or genomic DNA, respectively, obtained by cloning or produced synthetically by well known chemical techniques. DNA may be double- or single-stranded. Single-stranded DNA may comprise the coding or sense strand or the non-coding or antisense strand.

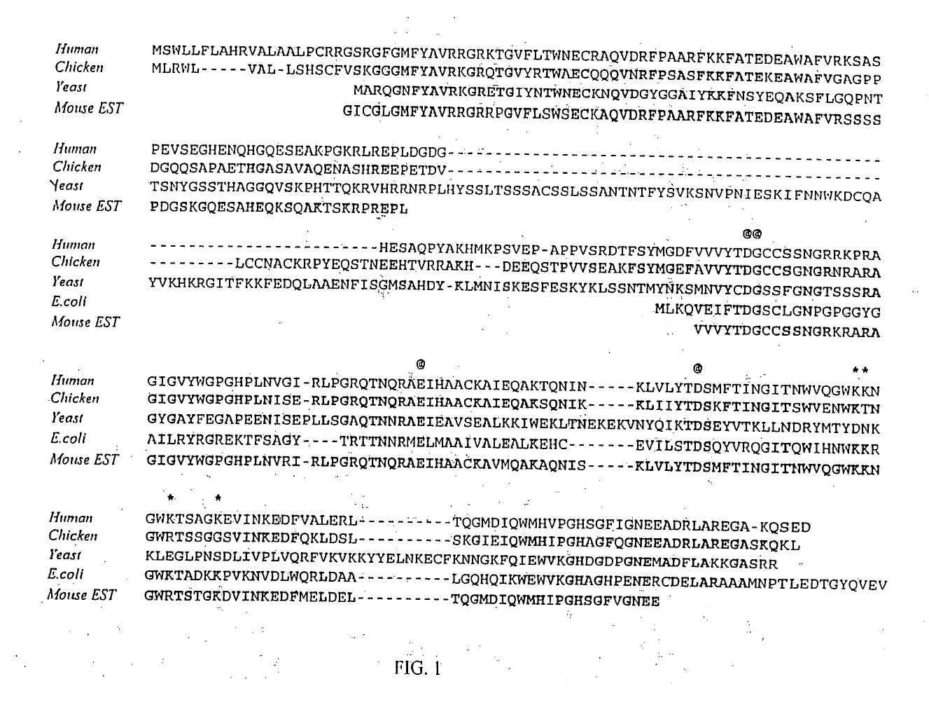

Methods of isolating a polynucleotide of the present invention via cloning techniques are well known. For example, to obtain the cDNA which encodes the polypeptide sequence provided herein as SEQ ID NO: 1, primers based on a search of the XREF database were used. A cDNA corresponding to the carboxy terminal portion of the protein was cloned by 3′ RACE. Positive clones were isolated by screening a human liver cDNA library with this cDNA. A 1131-nucleotide cDNA fragment encoding the full RNase HII protein sequence was identified and is provided herein as SEQ ID NO: 12. A single reading frame encoding a 299 amino acid protein (calculated mass: 33392.53 Da) was identified (shown in FIG. 1). This polypeptide sequence is provided herein as SEQ ID NO: 1.

In a preferred embodiment, the polynucleotide of the present invention comprises the nucleic acid sequence provided herein as SEQ ID NO: 12. However, as will be obvious to those of skill in the art upon this disclosure, due to the degeneracy of the genetic code, polynucleotides of the present invention may comprise other nucleic acid sequences encoding the polypeptide of SEQ ID NO: 1 and derivatives, variants or active fragments thereof.

Another aspect of the present invention relates to the polypeptides encoded by the polynucleotides of the present invention. In a preferred embodiment, a polypeptide of the present invention comprises the deduced amino acid sequence of human Type RNase HII provided in FIG. 1 as SEQ ID NO: 1. However, by “polypeptide” it is also meant to include fragments, mutants, derivatives and analogs of SEQ ID NO: 1 which retain essentially the same biological activity and/or function as human RNase HII. Alternatively, polypeptides of the present invention may retain their ability to bind to an antisense-RNA duplex even though they do not function as active RNase H enzymes in other capacities. In another embodiment, polypeptides of the present invention may retain nuclease activity but without specificity for the RNA portion of an RNA/DNA duplex. Polypeptides of the present invention include recombinant polypeptides, isolated natural polypeptides and synthetic polypeptides, and fragments thereof which retain one or more of the activities described above.

In a preferred embodiment, the polypeptide is prepared recombinantly, most preferably from the cDNA sequence provided herein as SEQ ID NO: 12. Recombinant human RNase H fused to histidine codons (his-tag; in the present embodiment six histidine codons were used) expressed in E. coli can be conveniently purified to electrophoretic homogeneity by chromatography with Ni-NTA followed by C4 reverse phase HPLC.

A recombinant human RNase HII (his-tag fusion protein) polypeptide of the present invention was expressed in E. coli and purified by Ni-NTA agarose beads followed by C4 reverse phase column chromatography. A 36 kDa protein (approx.) copurified with activity measured after renaturation. The presence of the his-tag was confirmed by Western blot analyses with an anti-penta-histidine antibody (Qiagen, Germany).

Renatured recombinant human RNase HII displayed RNase H activity. Incubation of purified renatured RNase HII protein with RNA/DNA duplex substrate for 60 minutes resulted in detectable cleavage of the substrate.

Accordingly, expression of large quantities of a purified human RNase HII polypeptide of the present invention is useful in characterizing the activities of this enzyme. In addition, the polynucleotides and polypeptides of the present invention provide a means for identifying agents which enhance the function of antisense oligonucleotides in human cells and tissues.

For example, a host cell can be genetically engineered to incorporate polynucleotides and express polypeptides of the present invention. Polynucleotides can be introduced into a host cell using any number of well known techniques such as infection, transduction, transfection or transformation. The polynucleotide can be introduced alone or in conjunction with a second polynucleotide encoding a selectable marker. In a preferred embodiment, the host comprises a mammalian cell. Such host cells can then be used not only for production of human RNase HII, but also to identify agents which increase or decrease levels of expression or activity of human RNase H in the cell. In these assays, the host cell would be exposed to an agent suspected of altering levels of expression or activity of human RNase H in the cells. The level or activity of human RNase H in the cell would then be determined in the presence and absence of the agent. Assays to determine levels of protein in a cell are well known to those of skill in the art and include, but are not limited to, radioimmunoassays, competitive binding assays, Western blot analysis and enzyme linked immunosorbent assays (ELISAs). Methods of determining increased activity of the enzyme, and in particular increased cleavage of an antisense-mRNA duplex can be performed in accordance with the teachings of Example 5. Agents identified as inducers of the level or activity of this enzyme may be useful in enhancing the efficacy of antisense oligonucleotide therapies.

The following nonlimiting examples are provided to further illustrate the present invention.

### EXAMPLES

### Example 1

### Cloning Human RNase HII by Rapid Amplification of 5′-cDNA End (5′ BRACE) and 3′-cDNA End (3′-RACE) of Human RNase HII

An internet search of the XREF database in the National Center of Biotechnology Information (NCBI) yielded 2 overlapping human expressed sequence tags (ESTs), GenBank accession numbers WO5602 and H43540, homologous to yeast RNase HII (RNH2) protein sequence (GenBank accession number Z71348; SEQ ID NO: 4 shown in FIG. 1), and its C. elegans homologue (accession number Z66524, of which amino acids 396-702 of gene TI3H5.2 correspond to SEQ ID NO: 3 shown in FIG. 1). Three sets of oligonucleotide primers hybridizable to one or both of the human RNase HII EST sequences were synthesized. The sense primers were AGCAGGCGCCGCTTCGAGGC (H1A; SEQ ID NO: 13), CCCGCTCCTGCAGTATTAGTTCTTGC (H1B; SEQ ID NO: 14) and TTGCAGCTGGTGGTGGCGGCTGAGG (H1C; SEQ ID NO: 15). The antisense primers were TCCAATAGGGTCTTTGAGTCTGCCAC (H1D; SEQ ID NO: 16), CACTTTCAGCGCCTCCAGATCTGCC (H1E; SEQ ID NO: 17) and GCGAGGCAGGGGACAATAACAGATGG (H1F; SEQ ID NO: 18). The human RNase HII 3′ cDNA derived from the EST sequence were amplified by polymerase chain reaction (PCR), using human liver Marathon ready cDNA (Clontech, Palo Alto, Calif.) as templates and H1A or H1B/AP1 (for first run PCR) as well as H1B or H1C/AP2 (for second run PCR) as primers. AP1 and AP2 are primers designed to hybridize to the Marathon ready cDNA linkers (linking cDNA insert to vector). The fragments were subjected to agarose gel electrophoresis and transferred to nitrocellulose membrane (Bio-Rad, Hercules Calif.) for confirmation by Southern blot, using a ³²P-labeled H1E probe (for 3′ RACE). The confirmed fragments were excised from the agarose gel and purified by gel extraction (Qiagen, Germany), then subcloned into a zero-blunt vector (Invitrogen, Carlsbad, Calif.) and subjected to DNA sequencing. The human RNase HII 5=cDNA from the EST sequence was similarly amplified by 5=RACE. The overlapping sequences were aligned and combined by the assembling programs of MacDNASIS v. 3.0 (Hitachi Software Engineering Co., America, Ltd.). The full length human RNase HII open reading frame nucleotide sequence obtained is provided herein as SEQ ID NO: 12. Protein structure and analysis were performed by the program MacVector v6.0 (Oxford Molecular Group, UK). The 299-amino acid human RNase HII protein sequence encoded by the open reading frame is provided herein as SEQ ID NO: 1.

### Example 2

### Screening of the cDNA Library and DNA Sequencing A Human Liver cDNA Lambda Phage Uni-ZAP Library

(Stratagene, La Jolla, Calif.) was screened using the 3=RACE products as specific probes. The positive cDNA clones were excised into pBluescript phagemid from lambda phage and subjected to DNA sequencing. Sequencing of the positive clones was performed with an automatic DNA sequencer by Retrogen Inc. (San Diego, Calif.).

### Example 3

### Northern Hybridization

Total RNA was isolated from different human cell lines (ATCC, Rockville, Md.) using the guanidine isothiocyanate method (21). Ten ìg of total RNA was separated on a 1.2% agarose/formaldehyde gel and transferred to Hybond-N+ (Amersham, Arlington Heights, Ill.) followed by fixing using UV crosslinker (Strategene, La Jolla, Calif.). The premade multiple tissue Northern Blot membranes were also purchased from Clontech (Palo Alto, Calif.). To detect RNase HII mRNA, hybridization was performed by using ³²P-labeled human RNase H II cDNA in Quik-Hyb buffer (Strategene, La Jolla, Calif.) at 68 EC for 2 hours. After hybridization, membranes were washed in a final stringency of 0.1×SSC/0.1% SDS at 60 EC for 30 minutes and subjected to auto-radiography.

RNase HII was detected in all human tissues examined (heart, brain, placenta, lung, liver, muscle, kidney and pancreas). RNase HII was also detected in all human cell lines tested (A549, Jurkat, NHDF, Sy5y, T24, MCF7, IMR32, HTB11, HUVEC, T47D, LnCAP, MRC5 and HL60) with the possible exception of NHDF for which presence or absence of a band was difficult to determine in this experiment. MCF7 cells appeared to have relatively high levels and HTB11 and HUVEC cells appeared to have relatively low levels compared to most cell lines.

### Example 4

### Expression and Purification of the Cloned RNase HII Protein

The cDNA fragment encoding the full RNase HII protein sequence was amplified by PCR using 2 primers, one of which contains a restriction enzyme NdeI site adapter and six histidine (his-tag) codons and a 22-base pair protein N terminal coding sequence, the other contains an XhoI site and 24 bp protern C-terminal coding sequence including stop codon. The fragment was cloned into expression vector pET17b (Novagen, Madison, Wis.) and confirmed by DNA sequencing. The plasmid was transfected into E. coli BL21(DE3) (Novagen, Madison, Wis.). The bacteria were grown in LB medium at 37EC and harvested when the OD₆₀₀ of the culture reached 0.8, in accordance with procedures described by Ausubel et al., (Current Protocols in Molecular Biology, Wiley and Sons, New York, N.Y., 1988). Cells were lysed in 8M urea solution and recombinant protein was partially purified with Ni-NTA agarose (Qiagen, Germany). Further purification was performed with C4 reverse phase chromatography (Beckman, System Gold, Fullerton, Calif.) with 0.1% TFA water and 0.1% TFA acetonitrile gradient of 0% to 80% in 40 minutes as described by Deutscher, M. P., (Guide to Protein Purification, Methods in Enzymology 182, Academic Press, New York, N.Y., 1990). The recombinant proteins and control samples were collected, lyophilized and subjected to 12% SDS-PAGE as described by Ausubel et al. (1988) (Current Protocols in Molecular Biology, Wiley and Sons, New York, N.Y.). The purified protein and control samples were resuspended in 6 M urea solution containing 20 mM Tris HCl, pH 7.4, 400 mM NaCl, 20% glycerol, 0.2 mM PMSF, 40 mM DTT, 10 ìg/ml aprotinin and leupeptin, and refolded by dialysis with decreasing urea concentration from 6 M to 0.5 M as well as DTT concentration from 40 mM to 0.5 mM as described by Deutscher, M. P., (Guide to Protein Purification, Methods in Enzymology 182, Academic Press, New York, N.Y., 1990). The refolded proteins were concentrated (10 fold) by Centricon (Amicon, Danvers, Mass.) and subjected to an RNase H activity assay as described in subsequent examples.

### Example 5

### RNase H Activity Assay

A ³²P-end-labeled 17-mer RNA, GGGCGCCGTCGGTGTGG (SEQ ID NO: 19) described by Lima, W. F. and Crooke, S. T. (Biochemistry, 1997 36, 390-398), was gel-purified as described by Ausubel et al. (Current Protocols in Molecular Biology, Wiley and Sons, New York, N.Y., 1988) and annealed with a tenfold excess of its complementary 17-mer oligodeoxynucleotide. Annealing was done in 10 mM Tris HCl, pH 8.0, 10 mM MgCl, 50 mM KCl and 0.1 mM DTT to form one of two different substrates: single strand (ss) RNA probe and full double strand (ds) RNA/DNA duplex. Each of these substrates was incubated with RNase HII protein samples (isolated as described in the previous example), or with the previously-cloned human RNase HI (Wu et al., 1999, J. Biol. Chem. 274, 28270-28278) at 37EC for 5 minutes to 60 minutes at the same conditions used in the annealing procedure and the reactions were terminated by adding EDTA in accordance with procedures described by Lima, W. F. and Crooke, S. T. (Biochemistry, 1997, 36, 390-398). The reaction mixtures were precipitated with TCA centrifugation and the supernatant was measured by liquid scintillation counting (Beckman LS6000IC, Fullerton, Calif.). An aliquot of the reaction mixture was also subjected to denaturing (8 M urea) acrylamide gel electrophoresis in accordance with procedures described by Lima, W. F. and Crooke, S. T. (Biochemistry, 1997, 36, 390-398) and Ausubel et al. (Current Protocols in Molecular Biology, Wiley and Sons, New York, N.Y., 1988). The gels were then analyzed and quantified using a Molecular Dynamics PhosphorImager. After 60 minutes, cleavage of the substrate RNA/DNA duplex was detectable.

### Example 6

### Characterization of Cloned Human RNase HII

The calculated molecular weight, estimated pI and amino acid composition of the cloned Rnase HII are shown in Table 1. The amino acid sequence is provided as SEQ ID NO: 1.

| | Human RNase | E. Coli | Yeast RNase |
|---|---|---|---|
| | HII | RNaseHII | HII |
| Calculated | 33392.53* | 21524.39 | 33923.36 |
| Molecular | | | |
| Weight | | | |
| Estimated pI | 4.94* | 7.48 | 9.08 |

| Amino acid | | | | | | | |
|---|---|---|---|---|---|---|---|
| composition | | No. | Percent | No. | Percent | No. | Percent |
| Nonpolar | A | 25 | 8.36 | 28 | 14.14 | 19 | 6.33 |
| | V | 26 | 8.70 | 16 | 8.08 | 24 | 8.00 |
| | L | 31 | 10.37 | 21 | 10.61 | 23 | 7.67 |
| | I | 8 | 2.68 | 11 | 5.56 | 16 | 5.33 |
| | P | 16 | 5.35 | 13 | 6.57 | 22 | 7.33 |
| | M | 5 | 1.67 | 6 | 3.03 | 11 | 3.67 |
| | F | 11 | 3.68 | 5 | 2.51 | 7 | 2.33 |
| | W | 5 | 1.67 | 1 | 0.51 | 4 | 1.33 |
| Polar | G | 17 | 5.69 | 14 | 7.07 | 12 | 4.00 |
| | S | 23 | 7.69 | 9 | 4.55 | 24 | 8.00 |
| | T | 16 | 5.35 | 6 | 3.03 | 19 | 6.33 |
| | C* | 6 | 2.01 | 1 | 0.51 | 3 | 1.00 |
| | Y | 10 | 3.34 | 5 | 2.53 | 14 | 4.67 |
| | N | 8 | 2.68 | 3 | 1.52 | 11 | 3.67 |
| | Q | 13 | 4.35 | 4 | 2.02 | 17 | 5.67 |
| Acidic | D | 19 | 6.35 | 8 | 4.04 | 19 | 6.33 |
| | E | 23 | 7.39 | 15 | 7.58 | 14 | 4.67 |
| Basic | K | 16 | 5.35 | 11 | 5.56 | 24 | 8.00 |
| | R | 18 | 6.02 | 12 | 6.06 | 15 | 5.00 |
| | H | 3 | 1.00 | 9 | 4.55 | 2 | 0.67 |

### Example 7

### Antisense Oligonucleotide Inhibition of RNase HII Expression

A series of antisense oligonucleotides were targeted to the human RNase HII polynucleotide sequence (SEQ ID NO: 12). These compounds were all 2′-O-methoxyethyl “gapmers” with an 8-nucleotide deoxy gap and a phosphorothioate backbone. Cytosine residues are 5-methyl cytosines. These compounds are shown in Table 2. The 2′-O-methoxyethyl (2′MOE) nucleotides are shown in bold.

TABLE 2

Antisense oligonucleotides targeted to human
RNase HII

| | | | | |
|---|---|---|---|---|
| ISIS | NUCLEOTIDE SEQUENCE | SEQ | TARGET | TARGET |
| NO. | (5′ → 3′) | ID NO: | SITE¹ | REGION |

| | | | | |
|---|---|---|---|---|
| 21946 | CGCCTCAGCCGCCACCACCA | 20 | 28 | 5′UTR |
| 21947 | CACAGGCGAACTCAGGCGAC | 21 | 90 | Coding |
| 21948 | GGACAATAACAGATGGCGTA | 22 | 188 | Coding |
| 21949 | CCCGCTCGCTCTCCAATAGG | 23 | 259 | Coding |
| 21950 | CCCAGCCGACAAAGTCCGTG | 24 | 304 | Coding |
| 21951 | CGGTGTCCACGAATACCTGG | 25 | 457 | Coding |
| 21952 | CGCGCCTGGTATGTCTCTGG | 26 | 485 | Coding |
| 21953 | GGTAGAGGGCATCTGCTTTG | 27 | 547 | Coding |
| 21954 | CCACCTTGGCACAGATGCTG | 28 | 583 | Coding |
| 21955 | CAGTTTCTCCACGAATTGCC | 29 | 627 | Coding |
| 21956 | TTTTGTCTTGGGATCATTGG | 30 | 681 | Coding |
| 21957 | AGCTGAACCGGACAAACTGG | 31 | 742 | Coding |
| 21958 | CCTCTTTCTCCAGGATGGTC | 32 | 775 | Coding |
| 21959 | ACTCCAGGCCGCGTTCCAGG | 33 | 913 | Coding |
| 21960 | CCTACGTGTGGTTCTCCTTA | 34 | 1003 | 3′UTR |
| 21961 | GCACACTCCCACCTTGCTTC | 35 | 1041 | 3′UTR |
| 21962 | CAAAAGGAAGTAGCTGGACC | 36 | 1071 | 3′UTR |

¹Location (position) of the 5′-most nucleotide of the oligonucleotide target site on the RNase HII target nucleotide sequence (SEQ ID NO: 12).

The oligonucleotides shown in Table 2 were tested by Northern blot analysis for their ability to inhibit expression of human RNase HII. Results are expressed in Table 3.

TABLE 3
Antisense inhibition of RNase HII expression

| ISIS | | | SEQ |
|---|---|---|---|
| NO. | % of control | % inhibition | ID NO: |
| 21946 | 50 | 50 | 20 |
| 21947 | 37 | 63 | 21 |
| 21948 | 38 | 62 | 22 |
| 21949 | 18 | 82 | 23 |
| 21950 | 32 | 68 | 24 |
| 21951 | 26 | 74 | 25 |
| 21952 | 11 | 89 | 26 |
| 21953 | 41 | 59 | 27 |
| 21954 | 23 | 77 | 28 |
| 21955 | 67 | 33 | 29 |
| 21956 | 37 | 63 | 30 |
| 21957 | 32 | 68 | 31 |
| 21958 | 62 | 38 | 32 |
| 21959 | 18 | 82 | 33 |
| 21960 | 8 | 92 | 34 |
| 21961 | 93 | 7 | 35 |
| 21962 | 63 | 37 | 36 |

ISIS 21946, 21947, 21948, 21949, 21950, 21951, 21952, 21953, 21954, 21956, 21957, 21959 and 21960 gave at least 50% inhibition of human RNase HII expression in this assay. Dose response curves for the two most active oligonucleotides in this experiment (ISIS 21952 and 21960; SEQ ID Nos 26 and 34, respectively) showed a 60% reduction of expression using either oligonucleotide at the lowest dose tested (50 nM) and approximately 70% reduction (ISIS 21952) and >80% reduction (ISIS 21960) at a concentration of 200 nM in A549 cells.

Additional oligonucleotides were targeted to human RNase HII (SEQ ID NO: 12). These are shown in Table 4. These compounds are either 2′-O-methoxyethyl “gapmers” with a phosphorothioate backbone or uniform 2′-O-methoxyethyls with a phosphorothiate backbone. Cytosine residues are 5-methyl cytosines. The 2′-O-methoxyethyl(2′MOE) nucleotides are shown in bold.

TABLE 4

Antisense oligonucleotides targeted to human
RNase HII

| ISIS | NUCLEOTIDE SEQUENCE | SEQ | TARGET | TARGET |
|---|---|---|---|---|
| NO. | (5′ → 3′) | ID NO: | SITE¹ | REGION |
| 113435 | AAACAATTTTAATGTCTGGG | 37 | 984 | 3′UTR |
| 113436 | AATTTTAATGTCTGGGTTGG | 38 | 980 | 3′UTR |
| 113437 | CCTTAAACAATTTTAATGTC | 39 | 988 | 3′UTR |
| 113449 | AAACAATTTTAATGTCTGGG | 37 | 984 | 3′UTR |
| 113450 | AATTTTAATGTCTGGGTTGG | 38 | 980 | 3′UTR |
| 113451 | CCTTAAACAATTTTAATGTC | 39 | 988 | 3′UTR |

1. An isolated human RNase HII polypeptide comprising SEQ ID NO: 1 or mutant form or active fragment thereof.

2. An isolated human RNase HII polypeptide encoded by the nucleotide sequence contained within ATCC Deposit No. PTA-2897 or mutant form or active fragment thereof.

3. A composition comprising a human RNase HII polypeptide and a pharmaceutically acceptable carrier.

4. An isolated polynucleotide encoding the human RNase HII polypeptide encoded by the nucleic acid sequence of the cDNA contained within ATCC Deposit No. PTA-2897, or mutant form or active fragment thereof.

5. The isolated polynucleotide of claim 4 which comprises SEQ ID NO: 12.

6. A vector comprising a nucleic acid encoding the human RNase H polypeptide encoded by the nucleic acid sequence of the cDNA contained within ATCC Deposit No. PTA-2897.

7. A host cell comprising the vector of claim 6.

8. A composition comprising a vector comprising a nucleic acid encoding a human RNase HII polypeptide and a pharmaceutically acceptable carrier.

9. A compound 8 to 50 nucleobases in length targeted to a nucleic acid molecule encoding a human RNase HII polypeptide, wherein said compound specifically hybridizes with and inhibits the expression of a human RNase HII polypeptide.

10. The compound of claim 9 which is an antisense oligonucleotide.

11. The compound of claim 10 wherein the antisense oligonucleotide has a sequence comprising at least an 8-nucleobase portion of SEQ ID NO: 20, 21, 22, 23, 24, 25, 26, 27, 28, 29, 30, 31, 32, 33, 34 or 36.

12. The compound of claim 10 wherein the antisense oligonucleotide comprises at least one modified internucleoside linkage.

13. The compound of claim 12 wherein the modified internucleoside linkage is a phosphorothioate linkage.

14. The compound of claim 10 wherein the antisense oligonucleotide comprises at least one modified sugar moiety.

15. The compound of claim 14 wherein the modified sugar moiety is a 2′-O-methoxyethyl sugar moiety.

16. The compound of claim 10 wherein the antisense oligonucleotide comprises at least one modified nucleobase.

17. The compound of claim 16 wherein the modified nucleobase is a 5-methylcytosine.

18. The compound of claim 10 wherein the antisense oligonucleotide is a chimeric oligonucleotide.

19. A composition comprising the compound of claim 9 and a pharmaceutically acceptable carrier or diluent.

20. The composition of claim 19 further comprising a colloidal dispersion system.

21. A method of inhibiting the expression of a human RNase HII polypeptide in cells or tissues comprising contacting said cells or tissues with the compound of claim 9 so that expression of the human RNase HII polypeptide is inhibited.

22. A method of treating an animal having a disease or condition associated with a human RNase HII polypeptide comprising administering to said animal a therapeutically or prophylactically effective amount of the compound of claim 9 so that expression of the human RNase HII polypeptide is inhibited.


SEQUENCE LISTING (39 sequences)

SEQ ID NO: 1 - protein, 299 aa, Homo sapien
MDLSELERDNTGRCRLSSPVPAVCRKEPCVLGVDEAGRGPVLGPMVYAICYCPLPRLADLEALLVADSLTLLESERERLFALMEDTDFVGWALDVLSPNLISTSMLGWVLYNLNSLSHDTATGLIQYALDQGVNVTQVFVDTVGMPETYQARLQQSFPGIEVTVLALADALYPVVSAASICALVARDQAVLLWQFVELLQDLDTDYGSGYPNDPLTLAWLLEHVEPVFGFPQFVRFSWRTAQTILELEAEDVIWEDSASENQEGLRLITSYFLNEGSQARPRSSHRYFLERGLESATSL

SEQ ID NO: 2 - protein, 128 aa, Mus sp.
MDLSELERDNTGRCRLSSPVPAVCLLEPCVLGVDEAGRGPVLGPMVYAICYCPLSRLADLEALLVADSLTLTENERERLFALMEEDGDFVGWALDVLSPNLISTSMLGRVLYNLNSLSHDTAAGLIQY

SEQ ID NO: 3 - protein, 307 aa, Caenorhabditis elegans
SLTVLYFIERMSLLCETERSLTWNNFGNGIPCVLGIDEAGRGPVLGPMVYAAAISPLDQNVELLNLGVDDSLALNEALREEIFNLMNEDEDIQQIIAYALRCLSPELISCSMLLRQLYSLNEVSHEAAITLIRDALACNVNVVEILVDTVGPLATYQALLELLFPGISICVTELADSLFPIVSAASIAALVTRDSRLRNWQFRELNILVPDAGYGSGYPGDPNTLLFLQLSVEPVFGFCSLVRSSWLTASTIVELRCVPGSWEDDEEEGLSQSLRMTSWMVPLNETEVVPLRNMEINLTLIVSTLFL

SEQ ID NO: 4 - protein, 307 aa, Saccharomyces cerevisiae
MVPPTVEASLESPYTLSYFSPVPSALLEQNDSPIIMGIDEAGRGPVLGPMVYAVAYSTQLYQDETIIPNYEFDDSLLLTDPIRRMLFSLIYQDNEELTQIGYATTCITPLDISRGMSLFPPTRNYNLNEQAHDVTMALIDGVILQNVLLSHVYVDTVGPPASYQLLLEQRFPGVLFTVALLADSLYCMVSVASVVALVTRDILVESLLRDPDEILGSGYPSDPLTVAWLLRNQTSLMGWPANMVRFSWQTCQTLLDDASLNSIPILWEEQYMDSRLNAAQLTLQLQLQMVALPVRRLRLRTLDNWYR

SEQ ID NO: 5 - protein, 198 aa, Escherichia coli
MIEFVYPHTQLVAGVDEVGRGPLVGAVVTAAVILDPARPIAGLNDSLLLSELRRLALYEEILELALSWSLGRAEPHEIDELNILHATMLAMQRAVAGLHIAPEYVLIDGNRCPLLPMPAMAVVLGDSRVPEISAASILALVTRDAEMAALDIVFPQYGFAQHLGYPTAFHLELLAEHGATEHHRRSFGPVLRALGLAS

SEQ ID NO: 6 - protein, 286 aa, Homo sapiens
MSWLLFLAHRVALAALPCRRGSRGFGMFYAVRRGRLTGVFLTWNECRAQVDRFPAARFLLFATEDEAWAFVRLSASPEVSEGHENQHGQESEALPGLRLREPLDGDGHESAQPYALHMLPSVEPAPPVSRDTFSYMGDFVVVYTDGCCSSNGRRLPRAGIGVYWGPGHPLNVGIRLPGRQTNQRAEIHAACLAIEQALTQNINLLVLYTDSMFTINGITNWVQGWLLNGWLTSAGLEVINLEDFVALERLTQGMDIQWMHVPGHSGFIGNEEADRLAREGALQSED

SEQ ID NO: 7 - protein, 286 aa, Homo sapiens
MSWLLFLAHRVALAALPCRRGSRGFGMFYAVRRGRLTGVFLTWNECRAQVDRFPAARFLLFATEDEAWAFVRLSASPEVSEGHENQHGQESEALASLRLREPLDGDGHESAEPYALHMLPSVEPAPPVSRDTFSYMGDFVVVYTDGCCSSNGRRRPRAGIGVYWGPGHPLNVGIRLPGRQTNQRAEIHAACLAIEQALTQNINLLVLYTDSMFTINGITNWVQGWLLNGWLTSAGLEVINLEDFVALERLTQGMDIQWMHVPGHSGFIGNEEADRLAREGALQSED

SEQ ID NO: 8 - protein, 286 aa, Homo sapiens
MSWFLFLAHRVALAALPCRRGSRGFGMFYAVRRGRLTGVFLTWNECRAQVDRFPAARFLLFATEDEAWAFVRLSASPEVSEGHENQHGQESEALASLRLREPLDGDGHESAEPYALHMLPSVEPAPPVSRDTFSYMGDFVVVYTDGCCSSNGRRRPRAGIGVYWGPGHPLNVGIRLPGRQTNQRAEIHAACLAIEQALTQNINLLVLYTDSMFTINGITNWVQGWLLNGWLTSAGLEVINLEDFVALERLTQGMDIQWMHVPGHSGFIGNEEADRLAREGALQSED

SEQ ID NO: 9 - protein, 286 aa, Homo sapiens
MSWLLFLAHRVALAALPCRRGSRGFGMFYAVRRGRLTGVFLTWNECRAQVDRFPAARFLLFATEDEAWAFVRLSASPEVSEGHENQHGRESEALASLRLREPLDGDGHESAEPYALHMLPSVEPAPPVSRDTFSYMGDFVVVYTDGCCSSNGRRRPRAGIGVYWGPGHPLNVGIRLPGRQTNQRAEIHAACLAIEQALTQNINLLVLYTDSMFTINGITNWVRGWLLNGWLTSAGLEVINLEDFVALERLTQGMDIQWMHVPGHSGFIGNEEADRLAREGALQSED

SEQ ID NO: 10 - protein, 299 aa, Homo sapiens
MDLSELERDNTGRCRLSSPVPAVCRLEPCVLGVDEAGRGPVLGPMVYAICYCPLPRLADLEALLVADSLTLLESERERLFALMEDTDFVGWALDVLSPNLISTSMLGRVLYNLNSLSHDTATGLIQYALDQGVNVTQVFVDTVGMPETYQAQLQQSFPGIEVTVLALADALYPVVSAASICALVARDQAVLLWQFVELLQDLDTDYGSGYPNDPLTLAWLLEHVEPVFGFPQFVRFSWRTAQTILELEAEDVIWEDSASENQEGLRLITSYFLNEGSQARPRSSHRYFLERGLESATSL

SEQ ID NO: 11 - protein, 285 aa, Mus sp.
MRWLLPLSRTVTLAVVRLRRGICGLGMFYAVRRGRRTGVFLSWSECLAQVDRFPAARFLLFATEDEAWAFVRSSSSPDGSLGQESAHEQLSQALTSLRPREPLGEGEELPEPGPLHTRQDTEPAAVVSLDTFSYMGESVIVYTDGCCSSNGRLRARAGIGVYWGPGHPLNVGIRLPGRQTNQRAEIHAACLAIMQALAQNISLLVLYTDSMFTINGITNWVQGWLLNGWRTSTGLDVINLEDFMELDELTQGMDIQWMHIPGHSGFVGNEEADRLAREGALQSED

SEQ ID NO: 12 - DNA, 1131 bp, Homo sapiens
cgcgcctgcagtattagttcttgcagctggtggtggcggctgaggcggcatggatctcagcgagctggagagagacaatacaggccgctgtcgcctgagttcgcctgtgcccgcggtgtgccgcaaggagccttgcgtcctgggcgtcgatgaggcgggcaggggccccgtgctgggccccatggtctacgccatctgttattgtcccctgcctcgcctggcagatctggaggcgctgaaagtggcagactcaaagaccctattggagagcgagcgggaaaggctgtttgcgaaaatggaggacacggactttgtcggctgggcgctggatgtgctgtctccaaacctcatctctaccagcatgcttgggtgggtcaaatacaacctgaactccctgtcacatgatacagccactgggcttatacagtatgcattggaccagggcgtgaacgtcacccaggtattcgtggacaccgtagggatgccagagacataccaggcgcggctgcagcaaagttttcccgggattgaggtgacggtcaaggccaaagcagatgccctctacccggtggttagtgctgccagcatctgtgccaaggtggcccgggaccaggccgtgaagaaatggcaattcgtggagaaactgcaggacttggatactgattatggctcaggctaccccaatgatcccaagacaaaagcgtggttgaaggagcacgtggagcctgtgttcggcttcccccagtttgtccggttcagctggcgcacggcccagaccatcctggagaaagaggcggaagatgttatatgggaggactcagcatccgagaatcaggagggactcaggaagatcacatcctacttcctcaatgaagggtcccaagcccgtccccgttcttcccaccgatatttcctggaacgcggcctggagtcagcaaccagcctctagcagctgcctctacgcgctctacctgcttccccaacccagacattaaaattgtttaaggagaaccacacgtaggggatgtacttttgggacagaagcaaggtgggagtgtgctctgcagccgggtccagctacttccttttggaaccttaaatagaatgggtgttggttgataaaaaaaaaaa

SEQ ID NO: 13 - DNA, 20 bp, Artificial sequence
  Synthetic
agcaggcgccgcttcgaggc

SEQ ID NO: 14 - DNA, 26 bp, Artificial sequence
  Synthetic
cccgctcctgcagtattagttcttgc

SEQ ID NO: 15 - DNA, 25 bp, Artificial sequence
  Synthetic
ttgcagctggtggtggcggctgagg

SEQ ID NO: 16 - DNA, 26 bp, Artificial sequence
  Synthetic
tccaatagggtctttgagtctgccac

SEQ ID NO: 17 - DNA, 25 bp, Artificial sequence
  Synthetic
cactttcagcgcctccagatctgcc

SEQ ID NO: 18 - DNA, 26 bp, Artificial sequence
  Synthetic
gcgaggcaggggacaataacagatgg

SEQ ID NO: 19 - DNA, 17 bp, Artificial sequence
  Synthetic
gggcgccgtcggtgtgg

SEQ ID NO: 20 - DNA, 20 bp, Artificial sequence
  Synthetic
cgcctcagccgccaccacca

SEQ ID NO: 21 - DNA, 20 bp, Artificial sequence
  Synthetic
cacaggcgaactcaggcgac

SEQ ID NO: 22 - DNA, 20 bp, Artificial sequence
  Synthetic
ggacaataacagatggcgta

SEQ ID NO: 23 - DNA, 20 bp, Artificial sequence
  Synthetic
cccgctcgctctccaatagg

SEQ ID NO: 24 - DNA, 20 bp, Artificial sequence
  Synthetic
cccagccgacaaagtccgtg

SEQ ID NO: 25 - DNA, 20 bp, Artificial sequence
  Synthetic
cggtgtccacgaatacctgg

SEQ ID NO: 26 - DNA, 20 bp, Artificial sequence
  Synthetic
cgcgcctggtatgtctctgg

SEQ ID NO: 27 - DNA, 20 bp, Artificial sequence
  Synthetic
ggtagagggcatctgctttg

SEQ ID NO: 28 - DNA, 20 bp, Artificial sequence
  Synthetic
ccaccttggcacagatgctg

SEQ ID NO: 29 - DNA, 20 bp, Artificial sequence
  Synthetic
cagtttctccacgaattgcc

SEQ ID NO: 30 - DNA, 20 bp, Artificial sequence
  Synthetic
ttttgtcttgggatcattgg

SEQ ID NO: 31 - DNA, 20 bp, Artificial sequence
  Synthetic
agctgaaccggacaaactgg

SEQ ID NO: 32 - DNA, 20 bp, Artificial sequence
  Synthetic
cctctttctccaggatggtc

SEQ ID NO: 33 - DNA, 20 bp, Artificial sequence
  Synthetic
actccaggccgcgttccagg

SEQ ID NO: 34 - DNA, 20 bp, Artificial sequence
  Synthetic
cctacgtgtggttctcctta

SEQ ID NO: 35 - DNA, 20 bp, Artificial sequence
  Synthetic
gcacactcccaccttgcttc

SEQ ID NO: 36 - DNA, 20 bp, Artificial sequence
  Synthetic
caaaaggaagtagctggacc

SEQ ID NO: 37 - DNA, 20 bp, Artificial sequence
  Synthetic
aaacaattttaatgtctggg

SEQ ID NO: 38 - DNA, 20 bp, Artificial sequence
  Synthetic
aattttaatgtctgggttgg

SEQ ID NO: 39 - DNA, 20 bp, Artificial sequence
  Synthetic
ccttaaacaattttaatgtc